(19) United States
(12) Patent Application Publication
(10) Pub. No.: US 2014/0171999 A1
(43) Pub. Date: Jun. 19, 2014

(54) SURGICAL INSTRUMENT INSERT AND SURGICAL INSTRUMENT SYSTEM

(71) Applicant: Mark Goldsmith, Leeds (GB)

(72) Inventor: Mark Goldsmith, Leeds (GB)

(73) Assignee: Surgical Innovations Limited, Leeds, Yorkshire (GB)

(21) Appl. No.: 14/112,874

(22) PCT Filed: Apr. 19, 2012
(86) PCT No.: PCT/GB2012/050855

§ 371 Date: Feb. 14, 2014

(30) Foreign Application Priority Data
Apr. 20, 2011 (GB) 1106672.7

(51) Int. Cl. A61B 17/3201

(52) U.S. Cl. CPC A61B 17/3201
USPC 606/174

(57) ABSTRACT

A retaining member for a surgical instrument insert is described. The instrument insert includes a retaining member which is free to move along the shaft before the insert is attached to the handle. The retaining member comprises a resilient member integrally formed with the retaining member. The resilient member can engage the surgical instrument insert shaft and act to prevent the retaining member falling off before the insert is attached to the handle. Because the resilient member is integrally formed the cost of manufacture is reduced. In another embodiment, a surgical instrument insert is provided wherein a proximal portion of the outer shaft is manufactured from an insulating material. The insulating material can be cheaper than the aluminum conventionally used. As a further advantage this also reduces the risk of electric shock and bums when used in an electrically live environment.

The present invention relates to a surgical instrument insert which may be attached and removed from a separate surgical instrument handle. The present invention also relates to a surgical instrument system including the insert and a surgical instrument handle. In particular, the present invention relates to features which allow connection of an outer shaft of a surgical instrument insert to a surgical instrument handle.

It is known to provide surgical instrument inserts that can be attached to and removed from a surgical instrument handle. Such systems are commonly used in minimally invasive or laparoscopic surgery. Typically the instrument insert comprises an outer shaft which is a hollow tube, an inner rod mounted within the outer shaft so that it can be moved relative to the outer shaft, and an operative portion attached to the distal end. The operative portion is connected to the outer shaft and the inner rod so that relative movement of the inner rod to the outer shaft causes actuation of the operative portion. A surgical instrument insert comprises means for connection to a surgical instrument handle at its proximal end. These means for connection connect the outer shaft and the inner member to the handle so that relative movement of the inner member to the outer shaft can be achieved by operation of the handle. An example of a surgical instrument system whereby surgical instrument inserts may be attached to surgical instrument handles is the Logic system commercially available from Surgical Innovations Limited, Leeds, UK.

In existing surgical instrument insert systems, for example the Logic system commercially available from Surgical Innovations Limited, the outer shaft of the insert is secured to a handle by a freely movable retaining member which is mounted around the outer shaft. The outer shaft also comprises an enlarged proximal end. The retaining member contains a screw thread at its proximal end and defines a receptacle for the enlarged element. To secure the outer shaft to the handle, the retaining member is slid to the proximal end and engaged with a screw thread of the handle. As the screw thread is tightened, the retaining member presses the enlarged portion against the handle, securing the other shaft in place.

Because the retaining member is free to move over the surgical instrument shaft it is possible that it may fall off the distal end of the surgical instrument insert during handling of the insert before it is attached to a handle. In that case, it is possible that the retaining member will contact non-sterile surfaces and the entire surgical instrument insert will have to be disposed or resterilised.

To reduce this problem, it is known to include a silicone O ring within the retaining member. For example, the retaining member may comprise an internal machined groove into which an O ring is fitted. The O ring narrows the diameter of the retaining member where it engages the shaft, increasing the friction between the retaining member and the instrument shaft and minimising the risk of the retaining member falling off the instrument insert during use.

Although the rubber O ring is effective, it significantly increases the cost of manufacturing the surgical instrument insert. The part count is increased and additional manufacturing steps are required to machine the groove and insert the O ring into the retaining member.

The enlarged portion is typically manufactured from aluminium. This has the advantage that it can be securely connected to the outer shaft proximal end by, for example, a screw fit. However, the use of aluminium increases the cost of manufacturing.

It would be desirable to provide a retaining member with a reduced manufacturing cost.

In some surgical procedures, the surgical instrument insert may be used in an environment with live electricity. In order to insulate the user of the surgical instrument insert, the shaft of the insert, which is typically made of metal, has an insulating cover. The integrity of the insulating covering is important: if it is broken the risk of electric shock and burns is increased. It would therefore be desirable that the retaining member can move over the shaft without risk of damaging any possible insulating covering provided on the shaft.

Use of an aluminium enlarged portion also creates difficulty when the instrument is used in a live environment. Although the insulating cover on the shaft can extend extremely close to the enlarged portion, the fact that the enlarged portion is made of metal still presents a risk of electric shock. This risk may be small because the insulation is only missing on the proximal end portion and in use the proximal end portion is covered by the retaining member, however, the risk is still present.

It would also be desirable to provide a surgical instrument insert with enhanced electrical insulation properties.

Accordingly, the present invention provides a retaining member for a surgical instrument insert which comprises a resilient member integrally formed with the retaining member. The resilient member can engage the surgical instrument insert shaft and act to prevent the retaining member falling off before the insert is attached to the handle. Because the resilient member is integrally formed the cost of manufacture is reduced.

In another aspect, the present invention provides a surgical instrument insert wherein a proximal portion of the outer shaft is manufactured from an insulating material. The insulating material can be cheaper than the aluminium conventionally used. As a further advantage this also reduces the risk of electric shock and burns when used in an electrically live environment.

According to a first aspect of the present invention, the surgical instrument insert comprises an outer shaft defining a longitudinal axis and having proximal and distal ends and a through bore extending between the proximal and distal ends; an inner rod mounted within the through bore and having proximal and distal ends; an operative portion connected to the distal end of the outer shaft and the distal end of the inner rod such that movement of the inner rod relative to the outer shaft results in actuation of the operative portion; and a retaining member mounted circumferentially around the outer shaft and moveable relative to the outer shaft, wherein the retaining member comprises at least one resilient member, wherein the at least one resilient member is integrally formed with the retaining member and the at least one resilient member engages the outer shaft.

The retaining member acts to retain the outer shaft on an attached surgical instrument. For example, the retaining member may include a screw thread or some form of snap-fit at its proximal end to engage a surgical instrument handle.

The at least one resilient member engages the outer shaft. Therefore, the resilient member will be at least partially deformed by its engagement with the outer shaft and thereby exert a force on the outer shaft. This force creates friction between the retaining member and the outer shaft, making it less likely that the retaining member will fall off the outer shaft before the insert is attached to a handle.

The reference that the resilient member is integrally formed with the retaining member is used to mean that the at least one resilient member is an integral part of the retaining member, i.e. the retaining member and the at least one resilient member are formed together in a single manufacturing process. This reduces manufacturing costs compared to prior art processes which involve additional manufacturing steps and use of additional parts to engage the outer shaft.

Preferably, there are a plurality of resilient members which are evenly spaced around the longitudinal axis. This provides a more even force around the outer shaft, and will also act to centre the retaining member on the outer shaft. In a preferable embodiment, four resilient members are provided which are spaced 90° apart along the longitudinal axis.

The at least one resilient member is preferably configured such that the retaining member can translate along the longitudinal axis and rotate about the longitudinal axis. This enables the retaining member to use a screw-fit with the handle. It can be translated to engage the screw thread and then rotated to tighten the fit.

In preferable embodiments, the resilient member has at least 4 degrees of freedom in its movement relative to the shaft. Preferably, these comprise translation along the longitudinal axis, rotation about the longitudinal axis and rotation about two other axes which are orthogonal with the longitudinal axis.

The at least one resilient member may comprise a cantilever arm extending from the retaining member and engaging the outer shaft. In this embodiment the force provided for the at least one resilient member is provided by bending the cantilever. In preferable embodiments, the cantilever may be aligned with the longitudinal axis. This configuration provides greater resistance to translation along the longitudinal axis than to rotation around it. This is preferable to reduce the risk of the retaining member falling off the instrument insert while also allowing it to be rotated easily to connect to the handle.

It is also preferable that the cantilever is attached at its proximal end, i.e. the end closer to the handle in use. This facilitates assembly of the insert because the presence of the enlarged portion means that the retaining member will be assembled from the distal end. This orientation may also provide greater resistance to movement in the distal direction and assist keeping the retaining member on the shaft.

Preferably, the at least one resilient member exerts a force on the outer shaft which is less than 4 N, more preferably less than 3 N. Keeping the force below this level helps to ensure that if the outer shaft is provided with insulation the force is not sufficient to tear or break the insulation as the retaining member moves over the outer shaft. In some embodiments the force may be at least 0.5 N, more preferably at least 1 N. This ensures that the force results in sufficient friction to reduce the risk of the retaining member falling off the outer shaft when the retaining member is not connected to a surgical instrument

Preferably, the retaining member is injection moulded. This allows the retaining member to be manufactured easily and at low cost. Any suitable material may be used, such as a medical grade plastics material.

In some embodiments, the surgical instrument further comprises an enlarged element attached to the proximal end of the outer shaft; and wherein the enlarged element comprises a first cylindrical portion having a first diameter longitudinally adjacent to a second cylindrical portion having a second diameter which is larger than the first diameter; the second portion is located proximal of the first portion along the longitudinal axis; and the enlarged element is an electrical insulator.

Optionally, the transition from the first to the second portion in the enlarged element may define a radial face which is substantially perpendicular to the longitudinal axis. The retaining member may engage this radial face to secure the outer shaft to an attached instrument handle. Alternatively, the transition from the first portion to the second portion may use a different profile. In that case, the retaining member will have a corresponding profile to engage the enlarged portion and secure it to a handle in use.

Preferably, the enlarged element defines an opening which is configured to fit over a proximal end portion of the outer shaft and the enlarged element is attached to the proximal end portion of the outer shaft by flaring the proximal end of the outer shaft within the opening. This enables a secure connection between the outer shaft and the enlarged element which is less dependent on the material characteristics. For example, it allows materials for the enlarged element which are not suitable in profile for forming a screw thread to attach to the proximal end of the outer shaft.

Preferably, the opening has a tapered or chamfered proximal end. This assists the flaring of the end of the outer shaft to provide a strong attachment.

The proximal end portion of the outer shaft may have a smaller outside diameter than the immediately adjacent portion of the outer shaft. This enables the enlarged element to fit over the proximal end of the outer shaft more easily. Advantageously, the first diameter may be substantially the same as the diameter of the outer shaft immediately adjacent the proximal end portion of the outer shaft. This means that the transition from the outer shaft to the enlarged element maintains a substantially constant diameter.

The proximal end portion of the outer shaft may further comprise at least one flat surface parallel to the longitudinal axis. This flat surface can assist with connection of an enlarged element when the orientation of the enlarged element relative to the outer shaft is important. In that case, a corresponding flat surface is defined within the opening of the enlarged element.

The surgical instrument insert may further comprise an electrically insulating covering at least on the outer shaft and the first portion of the enlarged element. This provides improved electrical insulation because the insulating covering extends beyond the interface between the outer shaft, which may be manufactured of metal and be conductive, and the insulating enlarged element.

According to another aspect of the present invention, the surgical instrument insert comprises an outer shaft defining a longitudinal axis and having proximal and distal ends and a through bore extending between the proximal and distal ends; an inner rod mounted within the through bore and having proximal and distal ends; an operative portion connected to the distal end of the outer shaft and the distal end of the inner rod such that movement of the inner rod relative to the outer shaft results in actuation of the operative portion; and an enlarged element attached to the proximal end of the outer shaft, wherein the enlarged element comprises a first portion having a first diameter longitudinally adjacent to a second portion having a second diameter which is larger than the first diameter; the second portion is located proximal of the first portion along the longitudinal axis; and the enlarged element is an electrical insulator.

This aspect provides the benefit of the improved enlarged element no matter what configuration of retaining member is used, for example it can be used with known retaining members having a machined groove and an O ring in the machine groove if required.

Preferably, the enlarged element defines an opening which is configured to fit over a proximal end portion of the outer shaft and the enlarged element is attached to the proximal end portion of the outer shaft by flaring the proximal end of the outer shaft within the opening.

The proximal end portion of the outer shaft may have a small outside diameter than the immediately adjacent portion of the outer shaft. The proximal end portion of the outer shaft may further comprise at least one surface parallel to the longitudinal axis.

The insert may further comprise an electrically insulating covering over at least the outer shaft and the first portion of the enlarged element.

According to a third aspect of the present invention, there is provided a surgical instrument system comprising a surgical instrument insert as discussed above, with or without the optional features also discussed, and a surgical instrument handle. In some embodiments the surgical instrument handle may provide a screw thread and the retaining member may comprise a corresponding screw thread at its proximal end.

Embodiments of the invention will now be described by way of example with reference to the accompanying drawings in which:

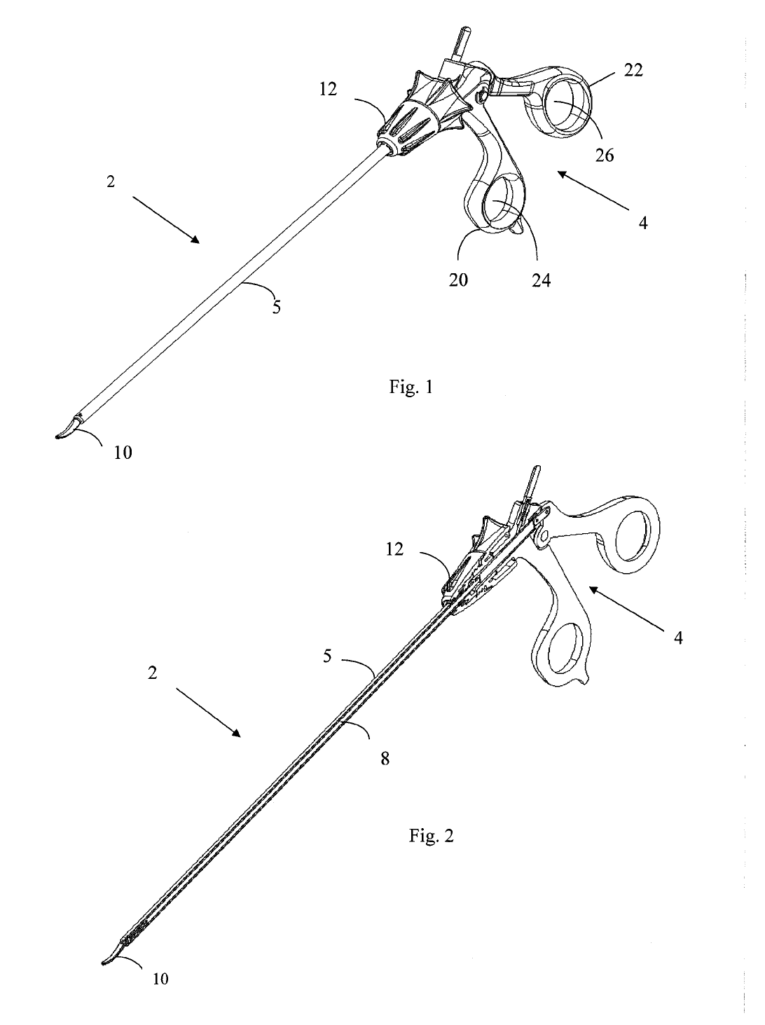

FIG. 1 is a perspective view of a surgical instrument insert according to the present invention attached to a surgical instrument handle;

FIG. 2 is a cross-section perspective view of FIG. 1 along a longitudinal axis;

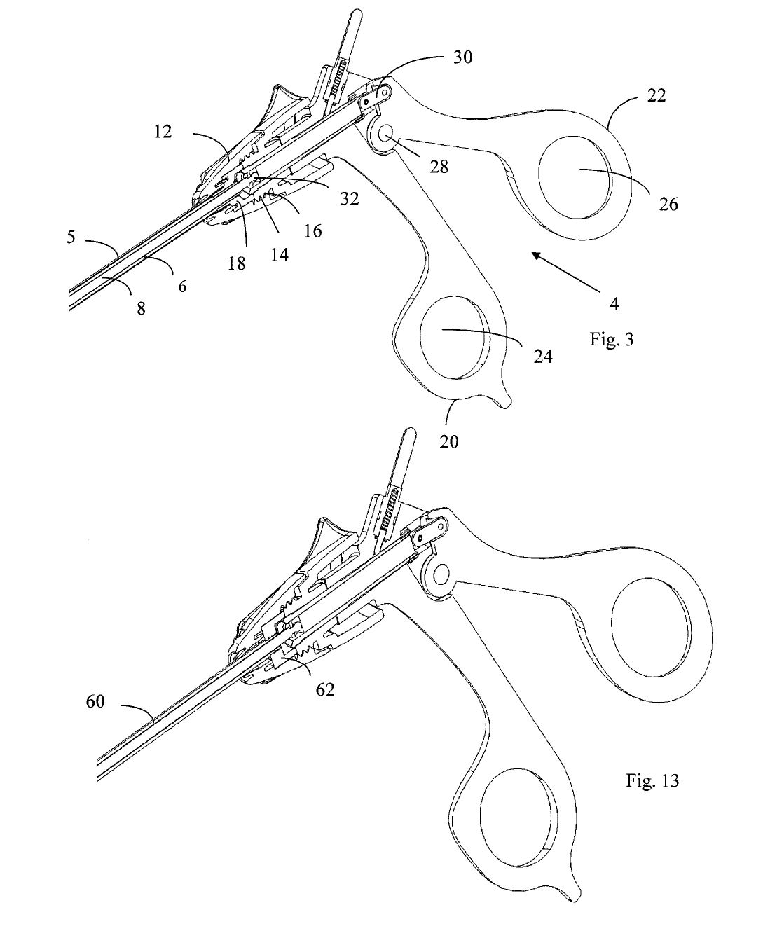

FIG. 3 depicts an enlarged partial cross-section view of FIG. 1;

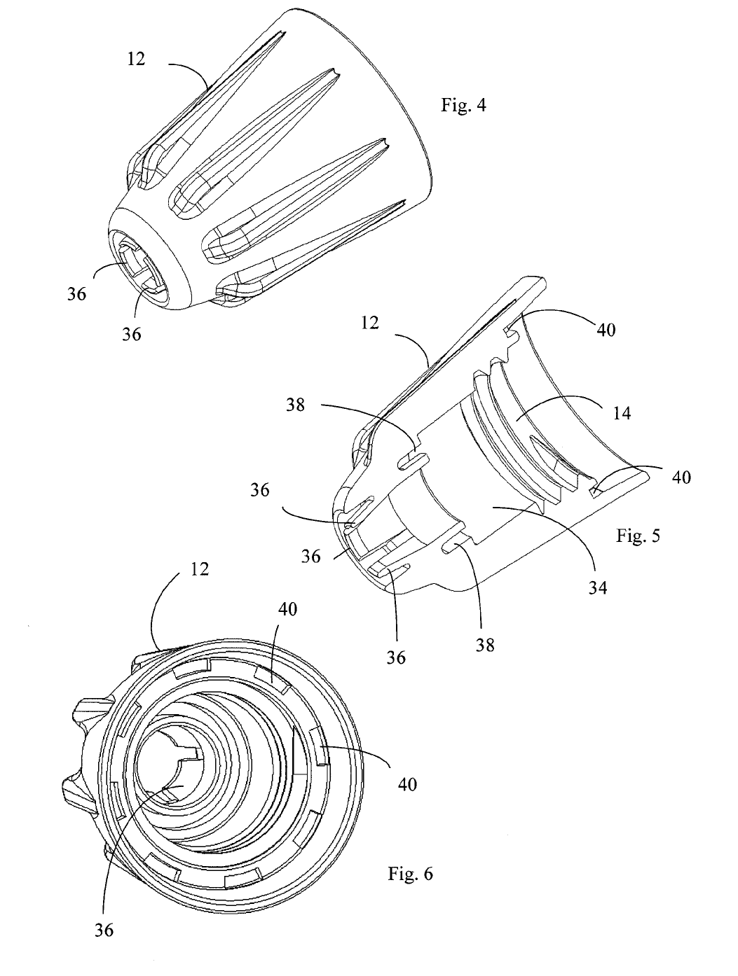

FIG. 4 depicts a perspective view of an improved retaining member depicted in cross-section in FIG. 3;

FIG. 5 depicts a cross-section perspective view of the retaining member of FIG. 4;

FIG. 6 depicts a rear perspective view of the retaining member of FIG. 4;

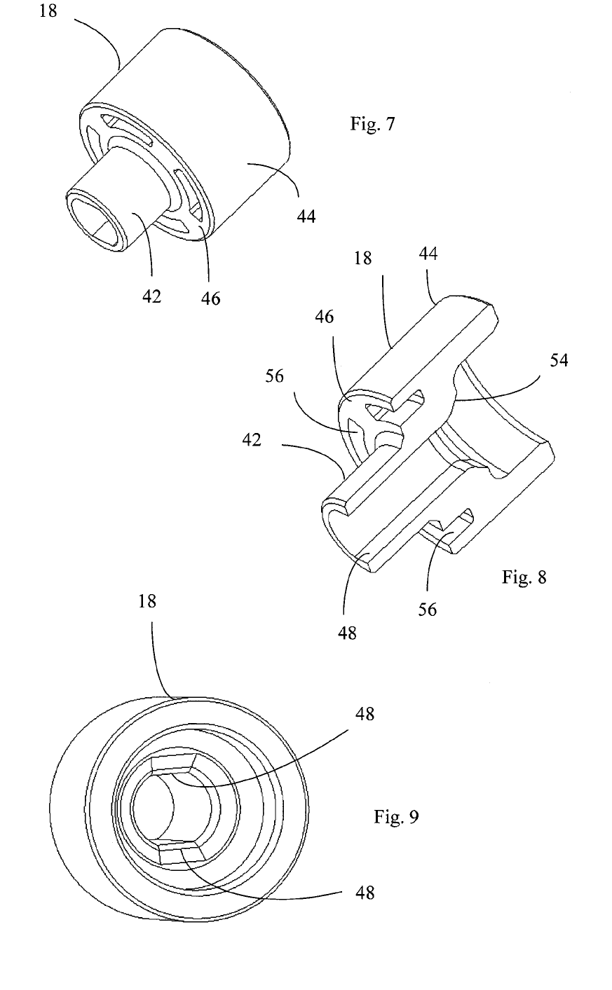

FIG. 7 depicts a perspective view of the improved enlarged element used in FIG. 3;

FIG. 8 is a cross-section perspective view of the enlarged element of FIG. 7;

FIG. 9 depicts a rear perspective view of the enlarged element of FIG. 7;

FIG. 10 depicts a perspective view of an improved instrument outer shaft used in FIG. 3;

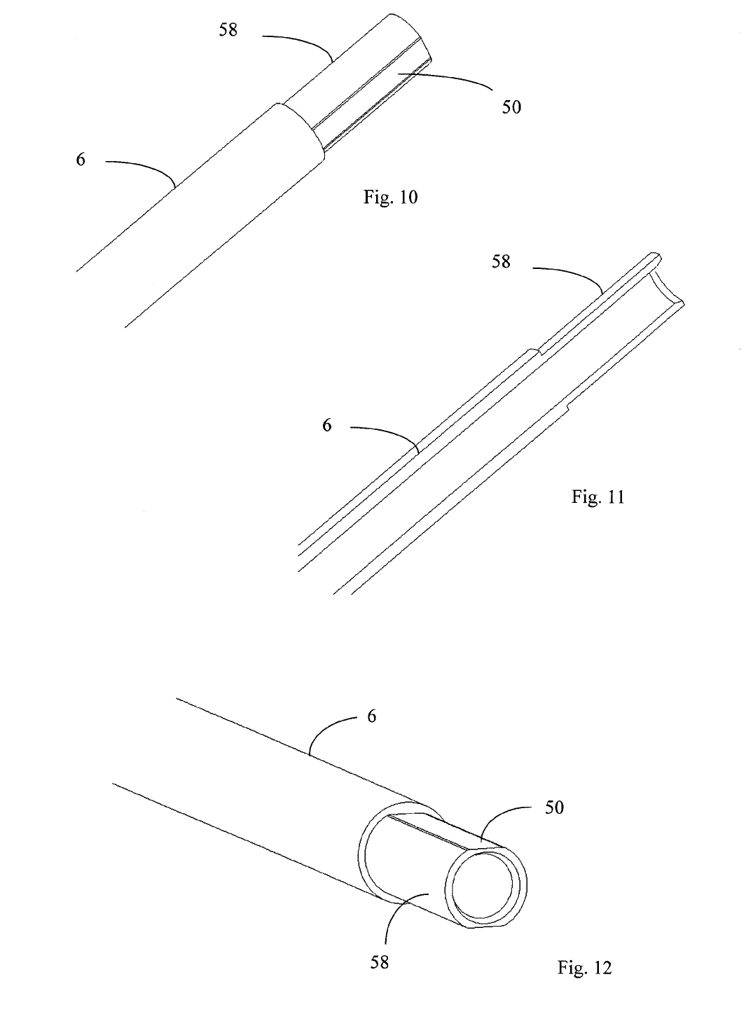

FIG. 11 depicts a perspective cross-section of the outer shaft of FIG. 10;

FIG. 12 depicts an alternative perspective view of the outer shaft of FIG. 10.

FIG. 13 depicts a cross-section of an alternative embodiment of an instrument insert attached to a surgical instrument handle.

Referring to FIGS. 1, 2 and 3, it can be seen that the surgical instrument insert 2 comprises an outer shaft 6 which is a hollow tube covered with an optional insulating covering 5. The outer shaft 6 defines a through bore in which an inner rod 8 is provided. At a distal end of the surgical instrument insert 2, an operative portion 10 is provided which is connected to both the inner rod 8 and the outer shaft 6. In this embodiment, the operative portion 10 is a surgical scissor, although in other embodiments any form of operative portion which is actuated by relative movement of inner rod 8 relative to outer shaft 6 can be used. Examples of alternative operative portions that may be used in other embodiments include, but are not limited to: a grasper, a dissector, a monopolar laparoscopic electrode as well as other surgical instruments for cutting tissue, manipulating tissue or placing and securing other medical devices.

The outer shaft 6 of the surgical instrument insert 2 is attached to the handle 4 by a retaining member 12. The retaining member 12 comprises a screw thread 14 at its proximal end which engages a corresponding screw thread 16 at the distal end of the handle. The retaining member 12 secures the outer shaft 6 in place on the handle by engaging an enlarged proximal end portion 18 of the outer shaft and pressing this against a distal end of the handle 4 by the engagement of screw threads 14 and 16.

The handle 4 comprises first and second pivoting arm members 20, 22. Both pivoting arm members 20, 22 comprise an opening 24, 26 for receiving a user's digit. The first and second pivoting arm members 20, 22 are connected for pivotal movement relative to each other about a joint 28. The first pivoting member 20 is secured to the outer shaft 6 of the instrument. The second pivoting member 22 is secured to the inner rod 8 of the instrument insert 2. A link member 30 converts the relative rotation of the first member 20 to the second member 22 into a translation along a longitudinal axis. The inner member 8 of the instrument insert 2 comprises an enlarged portion 32 which is engaged by the handle. Therefore, relative movement of the first member 20 to the second member 22 results in relative movement of the inner rod 8 relative to the outer shaft 6 along the longitudinal axis, actuating the operational portion.

Referring now to FIGS. 4-6, which show the retaining member 12 in isolation of the other components, the construction of the improved retaining member 12 of the present invention will now be described. Retaining member 12 comprises screw thread 14 at its proximal portion, as previously described. FIG. 5 also depicts how the retaining member 12 defines a receptacle 34 for receiving the enlarged proximal end portion 18 of the outer shaft. Retaining member 12 also defines an axial opening along its length, so that it can be fitted around the outer shaft 6.

At the distal end of the retaining member 12, four resilient members 36 are provided. Resilient members 36 are formed as cantilevers which extend at an angle to the axis of the retaining member 12, so that they narrow the effective diameter of the distal opening of the retaining member 12. In use, these resilient members 36 deform elastically to exert a force on the shaft. This force creates friction between the retaining member 12 and the outer shaft 6 which reduces the risk of the retaining member 12 sliding off the outer shaft 6 when it is not engaged with the screw thread 16 of the handle 4.

In this embodiment the retaining member 12 is formed as a single piece by injection moulding from a medical grade plastics material, for example polycarbonate, such as that commercially available from Bayer MaterialScience AG, Germany under the trade name Makrolon 2458. In order to reduce the effect of shrinkage during the injection moulding process, the retaining member 12 incorporates several sink portions 38, 40 to reduce the effect of shrinkage.

In this embodiment, resilient members 36 extend in a distal direction aligned with the longitudinal axis. In other words the centre line of each resilient member lies in a respective plane and each respective plane contains the longitudinal axis. The four resilient members are evenly spaced around the longitudinal axis so that the retaining member 12 is centred on the outer shaft. The number, length, angle and thickness of resilient members 36 may be adjusted according to known mechanical engineering principles to adjust the force which is applied on the instrument shaft 6. Thus, it is possible to control the force which is applied to the instrument shaft 6 by each resilient member 36. This is particularly advantageous when the outer shaft 6 is covered by insulating covering 5, which may be relatively soft and could be damaged by the pressure applied by the resilient members 36.

Moving on to FIGS. 7-9 the construction of the improved enlarged proximal portion 18 of the outer shaft 6 will now be described. The enlarged proximal portion 18 comprises a first portion 42 and a second portion 44 which are adjacent to each other along the longitudinal axis. The first portion 42 has a smaller diameter than the second portion 44. The first portion 42 preferably has a diameter substantially the same as the diameter of the outer shaft 6, however, this is not essential. The diameter of the second portion 44 is substantially the same as the diameter defined by receptacle 34 of the retaining member 12. The transition between the first portion 42 and the second portion 44 defines a face 46 which is perpendicular to the longitudinal axis. This face 46 engages a corresponding face in the receptacle 34 of the retaining member 12 when the outer shaft is attached to a surgical instrument handle 4. The enlarged element 18 defines an opening along its length, which partially contains the outer shaft 6 and the inner rod 8 when the surgical instrument insert 2 is assembled.

In order to ensure that the enlarged element 18 is aligned correctly with the outer shaft 6, the first portion 42 comprises flat surfaces 48 which are configured to engage corresponding flat surfaces 50 on the outer shaft. Flat surfaces 48 also improve the transfer of torque from the enlarged element 18 to the outer shaft 6. Torque may be required to be transferred without slippage via the enlarged element 18 if a rotational force about the longitudinal axis is applied to the enlarged element 18 through the surgical instrument handle 4.

The enlarged element 18 also comprises an internal angled or chamfered surface 54. This chamfered surface 54 allows the proximal end of the outer shaft 6 to be flared after it has been inserted into the enlarged element 18. This provides a secure connection between the enlarged element 18 and the outer shaft 6.

As with the retaining member 12, the enlarged element 18 is formed as a single piece by injection moulding plastics material. Again, sink portions 56 are provided to reduce the effects of shrinking during the injection moulding process. In this embodiment the enlarged element may be made from polycarbonate or any other suitable plastics material, but is preferably made from polyphenylsulfone, such as that commercially available from Port Plastics, Inc. under the trade name Radel PPSU R-5100.

The construction of the outer shaft 6 will now be described with reference to FIGS. 10, 11 and 12. The outer shaft 6 is hollow along its length to receive the inner member 8. It has a substantially constant diameter along its length, apart from a proximal portion 58 which is configured to be received in the enlarged element 18. In this embodiment proximal portion 58 has a reduced diameter compared to the remainder of the outer shaft 6. This enables the outer diameter of the first portion 42 of the enlarged element 18 to be substantially the same diameter as the remainder of the outer shaft 6. The reduced diameter portion 58 of the outer shaft 6 also comprises flat surfaces 50 to engage corresponding flat surfaces 48 in the enlarged element.

Outer shaft 6 is preferably manufactured from a metal or metal alloy, for example aluminium or aluminium alloy. This enables a connection between the outer shaft 6 and the enlarged proximal portion 18 by flaring the end of the outer shaft 6 over chamfered surface 54. Reduced diameter portion 58 has a length sufficient to engage the enlarged element 18 such that enlarged element 18 is unlikely to distort or bend at the point of its connection with the outer shaft 6.

In an alternative embodiment, depicted in enlarged cross-section view in FIG. 13, the construction of the instrument insert and handle is the same as described above, but with a modified outer shaft 60 and enlarged proximal portion 62. In this embodiment both outer shaft 60 and enlarged element 62 are manufactured from a metal or metal alloy, such as aluminium or aluminium alloy. The outer shaft 60 is attached to enlarged element 62 by a screw thread connection and may further be locked in place by flaring the excess material.

In another embodiment, not illustrated, the construction is the same as the first embodiment described, but the retaining member 12 is replaced with a conventional retaining member using a groove and a rubber O ring to engage the outer shaft. This embodiment uses the improved proximal end portion 18 described for the first embodiment and construction is otherwise the same as that embodiment.

According to the present invention, in an improved surgical instrument insert is provided which has reduced cost of manufacture. This is provided by the use of the improved retaining member and/or the enlarged proximal portion of the outer shaft. In addition, the enlarged proximal portion of the outer shaft enables improved electrical insulation of the outer shaft to be achieved in instruments where the outer shaft has an insulating covering.

1. A surgical instrument insert for connection to a surgical instrument handle, the surgical instrument insert comprising:
an outer shaft defining a longitudinal axis and having proximal and distal ends and a through bore extending between the proximal and distal ends;
an inner rod mounted within the through bore and having proximal and distal ends;
an operative portion connected to the distal end of the outer shaft and the distal end of the inner rod such that movement of the inner rod relative to the outer shaft results in actuation of the operative portion; and
a retaining member mounted circumferentially around the outer shaft and moveable relative to the outer shaft, wherein the retaining member comprises at least one resilient member, wherein the at least one resilient member is integrally formed with the retaining member and the at least one resilient member engages the outer shaft.

2. A surgical instrument insert according to claim 1, wherein there are a plurality of resilient members which are evenly spaced around the longitudinal axis.

3. A surgical instrument insert according to claim 1, wherein the at least one resilient member is configured such that the retaining member can translate along the longitudinal axis and rotate about the longitudinal axis.

4. A surgical instrument according to claim 1, wherein the at least one resilient member comprises a cantilever arm extending from the retaining member and engaging the outer shaft.

5. A surgical instrument according to claim 1, wherein the at least one resilient member exerts a force on the outer shaft which is less than 4 Newtons.

6. A surgical instrument according to claim 1, wherein the retaining member is injection moulded.

7. A surgical instrument according to claim 1, further comprising an enlarged element attached to the proximal end of the outer shaft; and wherein
the enlarged element comprises a first cylindrical portion having a first diameter longitudinally adjacent to a second cylindrical portion having a second diameter which is larger than the first diameter;
the second portion is located proximal of the first portion along the longitudinal axis; and
the enlarged element is an electrical insulator.

8. A surgical instrument insert according to claim 7, wherein the enlarged element defines an opening which is configured to fit over a proximal end portion of the outer shaft and the enlarged element is attached to the proximal end portion of the outer shaft by flaring the proximal end of the outer shaft within the opening.

9. A surgical instrument insert according to claim 8, wherein the proximal end portion of the outer shaft has a smaller outside diameter than the immediately adjacent portion of the outer shaft

10. A surgical instrument insert according to claim 8, wherein the proximal end portion of the outer shaft further comprises at least one flat surface parallel to the longitudinal axis.

11. A surgical instrument insert according to claim 7, further comprising an electrically insulating covering at least on the outer shaft and the first portion of the enlarged element.

12. A surgical instrument insert for connection to a surgical instrument handle, the surgical instrument insert comprising:
an outer shaft defining a longitudinal axis and having proximal and distal ends and a through bore extending between the proximal and distal ends;
an inner rod mounted within the through bore and having proximal and distal ends;
an operative portion connected to the distal end of the outer shaft and the distal end of the inner rod such that movement of the inner rod relative to the outer shaft results in actuation of the operative portion; and
an enlarged element attached to the proximal end of the outer shaft, wherein
the enlarged element comprises a first portion having a first diameter longitudinally adjacent to a second portion having a second diameter which is larger than the first diameter;
the second portion is located proximal of the first portion along the longitudinal axis; and
the enlarged element is an electrical insulator.

13. A surgical instrument insert according to claim 12, wherein the enlarged element defines an opening which is configured to fit over a proximal end portion of the outer shaft and the enlarged element is attached to the proximal end portion of the outer shaft by flaring the proximal end of the outer shaft within the opening.

14. A surgical instrument insert according to claim 13, wherein the proximal end portion of the outer shaft has a smaller outside diameter than the immediately adjacent portion of the outer shaft

15. A surgical instrument insert according to claim 13, wherein the proximal end portion of the outer shaft further comprises at least one flat surface parallel to the longitudinal axis.

16. A surgical instrument insert according to claim 12, further comprising an electrically insulating covering at least on the outer shaft and the first portion of the enlarged element.

17. A surgical instrument system comprising:
a surgical instrument insert according to claim 1; and
a surgical instrument handle.
